(19) United States
(12) Patent Application Publication
(10) Pub. No.: US 2018/0140269 A1
(43) Pub. Date: May 24, 2018

(54) X-RAY IMAGING

(71) Applicant: KONINKLIJKE PHILIPS N.V., EINDHOVEN (NL)

(72) Inventors: EWALD ROESSL, ELLERAU (DE); HEINER DAERR, HAMBURG (DE)

(21) Appl. No.: 15/569,832

(22) PCT Filed: May 6, 2016
(86) PCT No.: PCT/EP2016/060166

§ 371 Date: Oct. 27, 2017

(30) Foreign Application Priority Data
May 6, 2015 (EP) 15166499.2

(51) Int. Cl. A61B 6/00; A61B 6/03

(52) U.S. Cl. CPC A61B 6/484; A61B 6/032; A61B 6/4291; A61B 6/4241; A61B 6/482

(57) ABSTRACT

The intensity of an X-ray signal received at a detector after passing through an object of interest is a function of the attenuation, phase change, and scattering caused by the object of interest. In traditional X-ray systems, it was not possible to resolve these components. This application discusses an X-ray measurement technique which is insensitive to the variations in the interferometric pattern caused by phase differences in portions of the object of interest. Thus, received intensity measurements are caused only by attenuation and scattering components. By making two independent measurements of the object of interest using such a phase-invariant imager, the attenuation and scattering components may be separated, providing valuable extra information about the imaged object of interest arising from so-called “dark field” effects.

### FIELD OF THE INVENTION

The invention concerns an X-ray imaging system for imaging an object of interest, a method for X-ray imaging, a computer program element, a computer-readable medium, and a kit of parts for retrofitting a legacy X-ray scanner.

### BACKGROUND OF THE INVENTION

Conventional X-ray imaging involves sampling the intensity profile of an X-ray beam after it has passed through an object of interest, using traditional X-ray film, or a digital detector, for example. However, different materials in the object of interest affect the phase of an X-ray beam in different ways, providing another source of information about the internal structure of the object of interest. Historically, this information was lost. Phase-contrast X-ray imaging exploits the presence of phase changes caused to X-rays by imaged objects.

In a phase-contrast X-ray imaging setup, an X-ray source illuminates a phase grating, which establishes an interferometric pattern of X-ray maxima and minima beyond the phase grating, detected at an X-ray detector. A change in the phase in a portion of an X-ray beam incident on the phase grating will cause a related portion of an interferometric pattern to be displaced in the plane of the X-ray detector. A resolution of an X-ray detector is often not good enough to sample the interference pattern directly. Therefore, a movable analyzer grating is provided. A phase contrast imager samples the interference pattern by moving the analyzer grating a fixed number of steps across the plane of the X-ray detector, thus deriving information on the phase shift.

WO 2014/206841 concerns a phase-contrast imaging system. Such systems can, however, be further improved.

### SUMMARY OF THE INVENTION

Therefore, it would be advantageous to have an improved technique for X-ray imaging.

Towards this end, a first aspect of the invention provides an X-ray imaging system for imaging an object of interest. The system comprises an X-ray source, a phase grating, an analyzer grating, an X-ray detector, and a processing unit.

The X-ray source, the phase grating, the analyzer grating, and the X-ray detector are arranged in an optical path. The X-ray source is configured to apply X-rays to an object of interest positionable in the optical path.

The analyzer grating is provided in proximity to, or formed integrally with, the X-ray detector. The phase grating is configured to generate an interference pattern in the X-ray radiation comprising an intensity profile having an intensity peak with a full-width half-maximum distance which is narrow in comparison to a width of a transparent section of the analyzer grating, wherein the intensity peak of the interference pattern is incident on the X-ray detector through the transparent section of the analyzer grating.

The X-ray detector is configured to generate a first X-ray signal by measuring a first interference pattern and a second X-ray signal by independently measuring a second interference pattern. The interference patterns, which are generated by means of the phase grating, are indicative of an interaction of the X-ray radiation with an object of interest in the optical path. In generating the first and second X-ray signals, a difference in physical characteristics of the X-ray radiation used is being exploited.

The processing unit is configured to calculate an attenuation component and a dark-field component of the first and second interference patterns using the first and second X-ray signals.

According to this aspect of the invention, an X-ray imaging system is provided which does not require a sampling of the interference pattern using a phase stepping, as provided using a moving analyzer grating, or alternatively by moving the source grating or the focal spot of a phase-contrast configuration. Therefore, the mechanical complexity of a grating-based scanner can be reduced. In addition, a faster X-ray acquisition time is possible. This technique is also easier to apply to CT scanning, because phase-stepping is difficult to achieve in the rotating head of a CT scanner.

According to a second aspect of the invention, there is provided a method for X-ray imaging. The method comprises the following steps:

a) applying X-ray radiation to an object of interest using an X-ray source;
b) applying the X-ray radiation to a phase grating, wherein the phase grating is configured to generate an interference pattern in the X-ray radiation comprising an intensity profile having an intensity peak with a full-width half-maximum distance which is narrow in comparison to a width of a transparent section of the analyzer grating, wherein the intensity peak of the interference pattern is incident on the X-ray detector through the transparent section of the analyzer grating;
c) applying the X-ray radiation to an analyzer grating, wherein the analyzer grating is provided in proximity to, or formed integrally with, the X-ray detector;
d) generating a first X-ray signal by measuring a first interference pattern with the X-ray detector;
e) generating a second X-ray signal by measuring a second interference pattern indicative of an interaction of the X-ray radiation with an object of interest in the optical path; and
f) calculating an attenuation component, and a dark-field component, of the first and second interference patterns using the first and second X-ray signals.

According to a third aspect of the invention, there is provided a computer program element for controlling a system as described above, which, when being executed by a processing unit, is adapted to perform the method steps as described above.

According to a fourth aspect of the invention, a computer-readable medium having stored the program element previously described is provided.

According to a fifth aspect of the invention, a kit of parts for retrofitting a legacy X-ray scanner is provided.

The kit of parts comprises an X-ray detector having an analyzer grating in proximity to, or formed integrally with, the X-ray detector, a phase grating configured to generate an interference pattern in X-ray radiation, comprising an intensity profile having an intensity peak with a full-width half-maximum distance which is narrow in comparison to a width of a transparent section of the analyzer grating, wherein the intensity peak of the interference pattern is incident on the installed X-ray detector through a transparent section of the analyzer grating, and a computer-readable medium according to the description above.

The installation of the kit of parts to the legacy X-ray scanner enables the legacy X-ray scanner to calculate an attenuation component, and a dark-field component, of the X-rays.

Viewed another way, a concept of the invention is to measure the intensity profile using two independent measurements, that is, a physical characteristic of the X-ray radiation used in generating the interference patterns is different between the measurements. The attenuation and dark-field components of the intensity pattern can then be calculated. This is possible due to the phase-invariant detection behaviour of an X-ray detector, with an analyzer grating, and interference fringes with relatively thin intensity maxima. The interferometric device is thus insensitive to phase-shifts in the X-rays.

The present invention allows for useful application in a clinical environment such as a hospital. More specifically, the present invention is very suitable for application in imaging modalities such as mammography, diagnostic radiology, interventional radiology and computed tomography (CT) for the medical examination of patients. In addition, the presentation invention allows for useful application in an industrial environment. More specifically, the present invention is very suitable for application in non-destructive testing (e.g. analysis as to composition, structure and/or qualities of biological as well non-biological samples) as well as security scanning (e.g. scanning of luggage on airports).

In the following description, the term “an intensity profile” refers to a range of energies of a detected X-ray beam across the plane of an X-ray detector. Thus, in a pixelated X-ray detector, each pixel will record a different value for X-ray intensity when an inhomogeneous material is being imaged by the X-ray imaging system.

The intensity of the intensity profile detected at each pixel is a function of an attenuation component caused by absorption of the X-rays, a phase component caused by a phase change of the X-rays induced by the imaged material, and a scatter component caused by the small-angle scattering of X-rays inside the material. The intensity detected at each pixel is, therefore, a function of these three components. In the presence of a phase grating, the intensity profile across the X-ray detector plane will be in the form of an interferometric pattern, such as a Talbot carpet.

An intensity profile will have at least one “intensity maximum”. This is a point in the intensity profile which experiences the highest intensity. Of course, because an interferometric pattern is a repeating pattern, the intensity profile can also be considered to have a large plurality of maxima.

An “intensity peak” comprises an intensity maximum, and a certain distance either side of the peak before the energy in the peak has fallen away to some defined value. A peak may be defined by the “full-width half-maximum distance”.

The “full-width half-maximum distance” of a given mathematical function is the distance between two independent variables at which the dependent variable is equal to half of its maximum value. Thus, the distance between two points on either side of the intensity maximum which have an intensity half as great as that of the intensity maximum is a definition of the full-width half-maximum distance.

An “X-ray signal” is a series of pixel intensity values representing the intensity of the X-rays incident on an X-ray detector across the plane of the X-ray detector.

In the following description, the term “narrow in comparison to a width of a transparent section of the analyzer grating” means that the full-width half-maximum distance of the intensity peak is a small fraction of the width of a subsequent analyzer grating. One way of defining an intensity profile is through the use of the full-width half-maxima criterion.

In other words, an aspect of this invention exploits the fact that when narrow interference fringes are applied to an analyzer grating with transparent sections which are substantially wider than the interference fringes, a change in the phase of an imaged material will not be detected by an X-ray detector. This is possible because even though a phase change induced by an object under examination will cause portions of the interference pattern to move, the interference maxima carry the greatest share of the transmitted X-ray energy, and they can move around only inside one analyzer trench, because the interference maxima are very thin. The interference maxima do not collide with the grating bars of the analyzer grating, unless an extreme phase shift is experienced. Therefore, even quite substantial phase changes induced by a material under examination will not result in the interference maxima illuminating consecutive X-ray detector pixels simultaneously, and the interference patterns being generated essentially include components representing the attenuation and small angle scattering (dark-field component) of the X-ray radiation only.

In the absence of any phase-shift component signal variations, only two independent measurements (that is, measurements exploiting a difference in physicial characteristics of the X-ray radiation) of the intensity at the X-ray detector are required to disentangle the attenuation component and the scatter component of the incident X-rays. Thus, an imaging mechanism using an interferometer which does not require sampling with a stepped (mobile) analyzer grating is provided. Thus, advantageously, information about the dark-field component of the X-rays may be derived using a simpler and more efficient imaging system.

In an example of the X-ray imaging system according to the present invention, the physical characteristic being different is an energy level of the X-ray radiation. In this case, in particular, the X-ray detector may be an energy sensitive detector configured to generate the first X-ray signal by detecting a first photon energy, and to generate the second X-ray signal by detecting a second photon energy, wherein the first and second photon energies are mutually different.

In another example of the X-ray imaging system according to the present invention, use is made of a difference in a coherence of the X-ray radiation.

In this case, preferably, the X-ray imaging system is configured to generate each of the first X-ray signal and the second X-ray signal as composite signals, wherein with the first X-ray signal is based on a first measurement made with coherent X-rays, and a second measurement made with incoherent X-rays, and wherein the second X-ray signal is based on a third measurement made with coherent X-rays, and a fourth measurement made with incoherent X-rays.

In another example of the X-ray imaging system according to the present invention, the X-ray imaging system further comprises: a selectable X-ray scatterer positionable in the optical path and configurable into a first state in which the X-rays are coherent, and into a second state for interacting with the X-rays such that they become incoherent; wherein the first and third measurements are made with the selectable X-ray scatterer in the first state, and wherein the second and fourth measurements are made with the selectable Xray scatterer in the second state; and wherein the attenuation and dark-field components are calculated using the first, second, third, and fourth measurements.

In another example of the X-ray imaging system according to the present invention, the X-ray detector comprises a first section covered by an X-ray scatterer and a second section not covered by the X-ray scatterer; and the X-ray imaging system is configured to generate the first X-ray signal using the first section of the X-ray detector, and to generate the second X-ray signal using the second section of the X-ray detector.

In another example of the X-ray imaging system according to the present invention, the phase grating is configured to generate the interference pattern as having an intensity peak with a full-width half-maximum distance smaller than half of the period of the interference pattern.

In another example of the X-ray imaging system according to the present invention, the X-ray imaging system is selected from the group of: CT scanner, C-arm scanner, mammography scanner, tomosynthesis scanner, diagnostic X-ray scanner, pre-clinical imaging scanner, non-destructive testing scanner, or baggage security scanner.

In an example of the method of X-ray imaging method according to the present invention, the first X-ray signal is generated by detecting a first photon energy and the second X-ray signal is generated by detecting a second photon energy, wherein the first and second detected photon energies are mutually different.

In another example of the method of X-ray imaging method according to the present invention, the first X-ray signal is generated as a composite signal based on a first measurement made with coherent X-rays, and a second measurement made with incoherent X-rays; and the second X-ray signal is also generated as a composite signal based on a third measurement made with coherent X-rays, and a fourth measurement made with incoherent X-rays.

Another example of the method of X-ray imaging method according to the present invention, comprises the steps of: switching a selectable X-ray scatterer positionable in the optical path into a first state such that the X-rays are coherent; performing the first measurement; positioning the selectable X-ray scatterer in a second state in the optical path to interact with the X-rays such that the X-rays are incoherent; performing the second measurement; positioning the selectable X-ray scatterer in a first state out of the optical path such that the X-rays are coherent; performing the third measurement; positioning the selectable X-ray scatterer in a second state in the optical path to interact with the X-rays such that the X-rays are incoherent; and performing the fourth measurement; wherein the attenuation and dark-field components are calculated using the first, second, third, and fourth measurements.

In another example of the method of X-ray imaging method according to the present invention, the first X-ray signal is generated using a first section of the X-ray detector which is covered by an X-ray scatterer; and the second X-ray signal is generated using a second section of the X-ray detector not covered by the X-ray scatterer.

These and other aspects of the invention will become apparent from, and are elucidated, with reference to the embodiments described hereinafter.

### BRIEF DESCRIPTION OF THE DRAWINGS

Exemplary embodiments of the invention will be described with reference to the following drawings:

FIG. 1 illustrates an X-ray imaging system for imaging an object of interest according to a first aspect of the invention.

FIG. 2A illustrates a propagated wave phase profile resulting from a phase grating structure.

FIG. 2B illustrates an interference pattern caused by the phase grating structure of FIG. 2A.

FIG. 3 shows a portion of an X-ray detector.

FIG. 4A shows a portion of an X-ray detector with differing positions of interference maxima.

FIG. 4B shows an X-ray detector when imaging an object having microstructure.

FIG. 5 shows another example of an X-ray imaging system.

FIG. 6 shows a method according to a second aspect of the invention.

### DETAILED DESCRIPTION OF EMBODIMENTS

In the case of X-ray imaging, a significant amount of information is carried by the so-called “dark-field”, and this information provides useful information about an imaged object in a clinical situation. The dark-field is an image contrast characteristic which is formed by the mechanism of small-angle scattering of X-rays inside an object being imaged. Such scattering provides complementary, and otherwise inaccessible, structural information about an object to be imaged.

The intensity of an X-ray pattern is determined as a function of an attenuation component, a phase-change component, and a scatter component of the pattern.

Typically, the dark-field information is lost, because previously it has not easily been possible to resolve the dark-field component of the intensity profile. Conventionally (in differential phase-contrast imaging), an intensity profile is imaged by stepping an analyzer grating over a complete cycle of fringe phase realizations, and measuring the resulting intensity modulation observed due to the stepping of the analyzer grating (or movement of a source grating, or the X-ray source's focal spot).

From this modulation, a phase-change component of an X-ray beam can be determined. Such phase stepping is mechanically complicated. The technique is difficult to use in situations where the acquisition time is short. A mechanically complex machine will be more expensive. For CT imaging, for example, the rotation of the gantry during image acquisition forbids classical phase stepping for each angular view.

According to a first aspect of the invention, an X-ray imaging system 10 for imaging an object of interest is provided. The system comprises an X-ray source 12, a phase grating 14, an analyzer grating 16, an X-ray detector 18, and a processing unit 20.

The X-ray source 12, the phase grating 14, the analyzer grating 16, and the X-ray detector 18 are arranged in an optical path 22. The X-ray source 12 is configured to apply X-rays to an object of interest 28 positionable in the optical path 22. The analyzer grating 16 is provided in proximity to, or formed integrally with, the X-ray detector 18. It will be understood that the object of interest 28 is removable, and is not part of the invention.

The phase grating 14 is configured to generate an interference pattern in the X-ray radiation comprising an intensity profile having a maximum with a full-width half-maximum distance which is narrow in comparison to a width of a transparent section of the analyzer grating. The intensity maximum is incident on the X-ray detector 18 through the transparent section of the analyzer grating. Typically, a Talbot interferometer is applied, using a special grating capable of generating suitable X-ray interferometric patterns with a plurality of fine interference maxima.

According to an embodiment of the invention, the phase grating is configured to generate a Talbot carpet.

The X-ray detector 18 is configured to generate a first X-ray signal by measuring a first interference pattern. The X-ray detector is configured to generate a second X-ray signal by measuring a second interference pattern. The interference patterns are indicative of an interaction of the X-ray radiation with an object of interest in the optical path. The processing unit 20 is configured to calculate an attenuation component, and a dark-field component, of the first and second interference patterns using the first and second X-ray signals. Preferably, in generating the first and second X-ray signals, different physical properties of the X-ray radiation are being exploited.

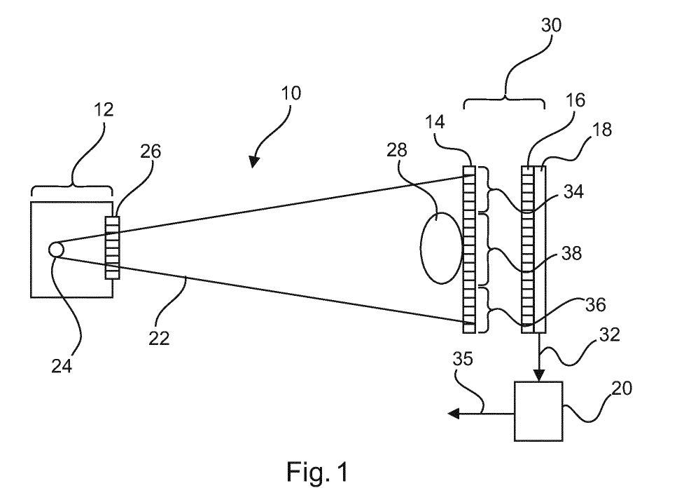

FIG. 1 shows an example of the system 10 according to a first aspect of the invention. The X-ray source 12 is shown comprising, for example, a rotating anode X-ray tube 24. Radiation emitted from the X-ray tube is incoherent. Interferometry assumes the use of coherent radiation. Therefore, when an X-ray rotating tube is used as the source 24, coherent X-rays are provided by shining the X-ray beam through a source grating 26 designed to provide coherent radiation. Of course, there are methods of providing coherent X-ray radiation without using a source grating.

According to an alternative embodiment, the X-ray source is a synchrotron or a free-electron laser.

When a coherent source is available, the source grating 26 could, optionally, be omitted. The optical path 22 lies in a line between the X-ray source 12 and the phase grating 14. Beyond the phase grating 14 is the analyzer grating 16 which is provided in close proximity to, or formed integrally with, the X-ray detector 18.

The X-ray detector 18 comprises a plurality of pixels which emit an electrical signal proportional to an intensity of incident X-ray light on the pixel. Alternatively, the X-ray detector 18 may be an energy-resolving photon counting detector, capable of resolving photons of different energies into different energy bins.

When the X-ray source 12 is energized, an X-ray beam is incident on the source grating 26. The object of interest 28 is illuminated, and the phase grating 14 establishes an interference pattern subsequent to the phase grating 14 in the X-ray radiation. Thus, the space defined by the bracket 30 may be considered an interferometer. Fringes of the interference pattern will be incident on the analyzer grating 16. A section of the analyzer grating comprises an X-ray blocking material, such as gold, which blocks incident sections of the interference pattern. Conversely, a transparent portion of the analyzer grating will enable X-ray radiation incident at that location to continue into the X-ray detector 18, and to be detected.

The processing unit 20 is configured to collect a plurality of signals 32 from the X-ray detector, which are collected and pre-processed using readout electronics 32. The processing unit 20 calculates the component of the received of the first and second interference patterns due to attenuation, and/or scattering, respectively. The attenuation and scattering components are then output to a subsequent system 35. The subsequent system is a storage device, a viewing monitor, or a communication connection, for example.

The interference pattern obtained at the regions of the interferometer encompassed by brackets 34 and 36 does not comprise a phase disturbance caused by the object of interest 28, because the X-ray has not passed through the object of interest. In contrast, the interference pattern in the direct optical path of the object of interest 28 represented by bracket 38 will be translated across the X-ray detector plane 18.

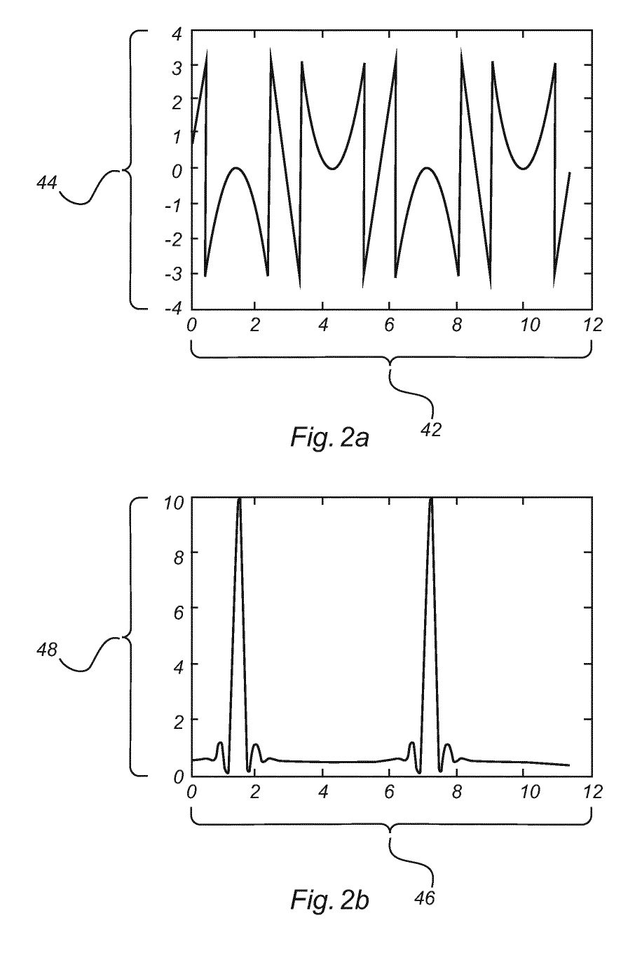

Turning now to FIG. 2A, further particulars of the phase grating 14 according to an embodiment of the invention are discussed. The phase grating 14 is configured to generate an interference pattern in the X-ray radiation comprising an intensity profile having an intensity peak with a full-width half-maximum distance which is narrow in comparison to a width of a transparent section of the analyzer grating.

To generate an intensity profile having an intensity peak with a full-width half-maximum distance which is narrow in comparison to a transparent section of the analyzer grating, the phase grating should be designed to generate fine interference fringes, and the analyzer grating should be designed to have a wider duty cycle than is typical. FIG. 2A shows a propagated wave phase profile associated with a phase grating structure designed to generate interference patterns with intensity maxima with full-width half-maxima (FWHM) significantly smaller than the half of the periodicity of the patterns.

International publication number WO 2012/104770 A2 discusses the design of such phase gratings as deflection structure plates.

In FIG. 2A, the x-axis 42 represents a transverse dimension across a plane of the phase grating 16. The y-axis 44 illustrates an X-ray phase difference (ranging between +π to −π radians) at certain points on a transverse dimension across the grating.

FIG. 2B shows a propagated wave intensity profile across the plane of an X-ray detector when the phase grating structure of FIG. 2A is applied as the phase grating 16, and when no analyzer grating is present.

The x-axis 46 in FIG. 2B illustrates the transverse dimension in micrometers across a typical interference pattern. The y-axis 48 illustrates the normalized X-ray intensity across a detector plane in an interferometer. As shown, the phase grating structure illustrated in FIG. 2A results in a propagated wave intensity profile at the detector having two peaks with a form approaching that of a squared sinc function. Therefore, the interference fringes are much finer than the usual sinusoidal wave intensity profile expected in conventional stepped phase-contrast systems.

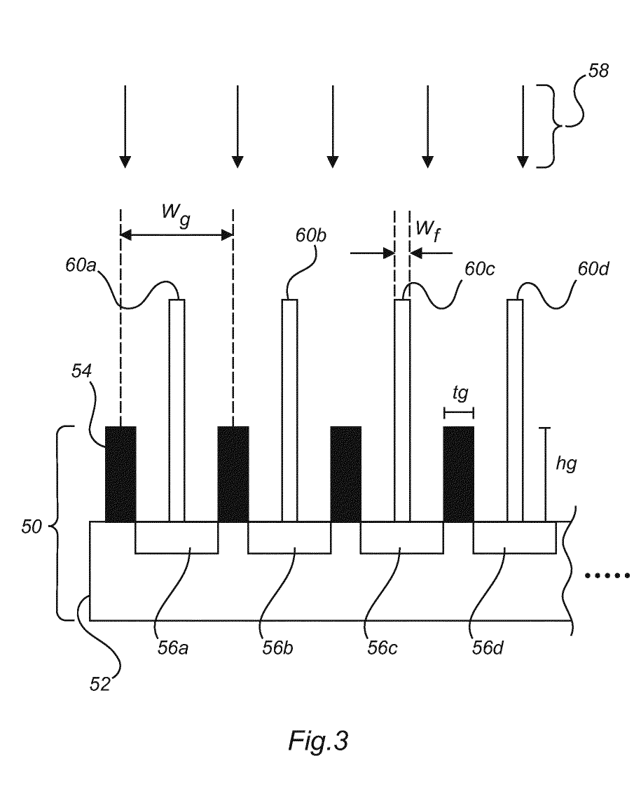

Turning to FIG. 3, an X-ray detector arrangement 50 is shown. The X-ray detector arrangement 50 comprises a silicon wafer 52 in which pixels 56 of an X-ray detector 18 are fabricated, and a plurality of analyzer grating lines 54. The analyzer grating lines are made from a dense material which absorbs X-rays. For example, the analyzer grating can be made from gold.

The analyzer grating is provided in proximity to, or formed integrally with, the X-ray detector 18 (fabricated in the silicon wafer 52). Therefore, in the embodiment illustrated in FIG. 3, the analyzer grating line 54 is attached directly to the silicon wafer 52, for example, because it has been deposited in a deposition process. Alternatively, the analyzer grating line 54 may be arranged on another X-ray transparent material, and held proximately to the silicon wafer 52.

The silicon wafer 52 comprises a plurality of X-ray detector pixels 56a, 56b, 56c, 56d. When the pixels 56 are exposed to X-ray radiation, they emit an electrical signal which may be detected by readout electronics, and sent for further processing. The magnitude of the electrical signal transmitted is proportional to the intensity of the X-rays incident on each pixel.

Alternatively, energy resolving detector pixels (and accompanying circuitry) can identify photons with different energies and allocate them to specific energy bins.

In FIG. 3, the analyzer grating has a pitch W_{g}, a grating line thickness t_{g}, and a height h_{g}. A portion of the silicon wafer not covered by one of the analyser grating lines 54 is considered to be a transparent grating portion, allowing the unattenuated passage of X-ray radiation, compared to the analyser grating lines 54. The pitch W_{g} is the width of the grating line plus the width of a transparent grating portion. X-rays passing through a phase grating (not shown) are incident on the analyzer grating lines 54. The X-ray wave front is illustrated by arrows 58.

With the use of a phase grating according to an embodiment of the invention, fine interference fringes represented by 60a, 60b, 60c, and 60d are, respectively, incident on the X-ray detector pixels 56a, 56b, 56c, and 56d, through the transparent grating portions.

Each transparent portion of the analyzer grating 54 is aligned with a respective detector pixel 56a, 56b, 56c, 56d,

In the example shown, the fine interference fringes have an intensity profile having a full-width half-maximum distance which is narrow in comparison to a width of a transparent section of the analyzer grating. Therefore, substantially all of the energy in the interference fringes passing through the transparent grating portions will be resolved by the X-ray detector, allowing for the usual X-ray detector conversion losses.

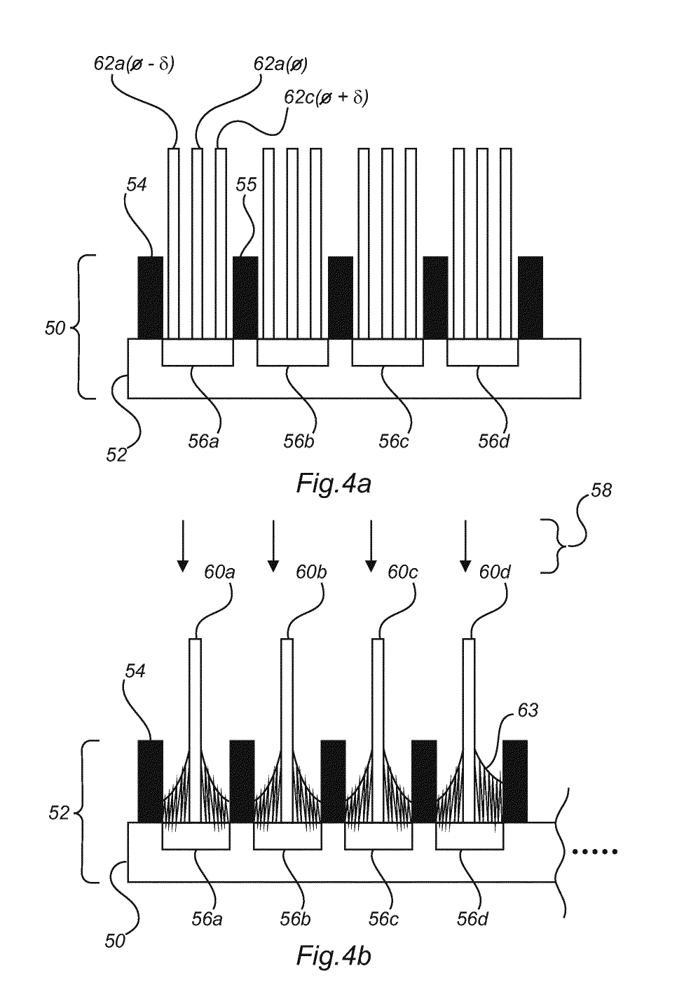

FIG. 4A shows the X-ray detector assembly 50 resembling that of FIG. 3 comprising the same analyzer grating lines 54 and X-ray detector 52 on a silicon wafer. In this situation, the effects of three different interference profiles are illustrated on the same drawing. As is known, a material portion causing a phase difference in an X-ray in the optical path 28 will result, subsequently to the phase grating 14, in a transverse movement of the interference pattern on the analyzer plane.

Thus, for example, an interference maximum 62b, indicates the normal position of an interference maximum at a reference phase angle φ radians. The position of an interference fringe 62a at the left extremity of the pixel illustrates a phase shift in a portion of the interference pattern of φ−δ radians. An interference fringe 62c illustrates the position of an interference fringe with a phase-shift of φ+δ radians. Such movements could be caused by a material transition in the object of interest 28, from soft tissue to bone, for example.

It can, therefore, be seen that as phase-shifts are experienced in portions of the optical path, relatively narrow interference maxima 62 will drift around in the trenches of the analyzer grating 54. Because each transparent portion of the analyzer grating 54 is aligned with one of the detector pixels 56a, 56b, 56c, 56d, it is clear that even with phase-shifts as small as φ−δ, or as large as φ+δ, the interference maxima will not collide with the analyser grating lines 54, and the same detector pixel will be illuminated for a wide range of phases.

Should a phase-shift greater than φ−δ or φ+δ be experienced by the wave front, the interference fringes 62 will collide with the grating 54 or 55, for example. However, the grating dimensions can be designed so that for most phase-shifts experienced due to an object of interest for a specific application area of the X-ray imaging system, the X-ray detector will be substantially phase invariant.

In other words, the arrangement of a phase grating generating an intensity profile having a full-width half-maximum distance which is narrow in comparison to a width of a transparent section of the analyzer grating 54 enables the phase component (due to variations in the material homogeneity of the object of interest) to be removed from the intensity profile detected by the X-ray detector 18.

The remaining components of the intensity profile result from the attenuation by the object of interest, and from scattering of the X-ray wave front by microstructures in the imaged material. Such scattering is referred to as dark-field scattering. Because the phase variance caused by the material is removed by such a combination of phase grating and analyzer grating, to separate the attenuation and dark-field components at least two independent measurements of the object of interest are made. Then, the attenuation component and the dark-field component of the X-rays may be calculated. Methods of performing such independent measurements will be discussed subsequently.

FIG. 4B shows a situation where the object of interest in the optical path illuminated by the X-ray source 12 is the object of interest 28 containing a microstructure. Typical microstructures are, for example, the fine matrices of bone found on the inside of mammal bones, having a matrix repetition on the order of micometres.

The intensity modulation observed at the analyzer grating shows how the interferometer as previously described is sensitive to a disturbance of flatness of the wave front induced by a microstructure. It can be seen that interference fringes caused by refraction cause a broadening 63 in the spectral characteristic. More and more intensity will be absorbed in the analyzer grating lines 54 of the analyzer grating, which will correspond to a reduction in intensity absorbed at each of the pixels 56a, 56b, 56c, 56d.

As previously stated, in order to distinguish the reduction of the measured intensity by attenuation and scattering, at least two distinct measurements are required, without movement of the analyzer grating 16 is necessary.

Thus, in an embodiment of the invention, the analyzer grating is a static grating.

This compares favourably to the requirements of a differential phase contrast imaging system, in which about eight mechanical phase steps of an analyzer grating must be made to determine X-ray intensity profile, implying a time delay and mechanical complexity.

As discussed above, the phase grating 14 is configured to generate an interference pattern in the X-ray radiation comprising an intensity profile having an intensity peak with a full-width half-maximum distance which is narrow in comparison to a width of a transparent section of the analyzer grating.

What constitutes a narrow full-width half-maximum distance, and the grating sizes able to achieve this, will now be discussed in more detail.

According to an embodiment of the invention, the X-ray imaging system 10 is provided as described previously, wherein the phase grating 14 is configured to provide the interference pattern as an interference pattern with a full-width at half-maximum smaller than half of the period of the interference pattern.

According to an embodiment of the invention, the X-ray imaging system 10 is provided as described previously, wherein the phase grating 14 is configured to provide the interference pattern as an interference pattern with a full-width at half-maximum smaller than half of the width of the analyzer pitch W_{g}.

According to an embodiment of the invention, the X-ray imaging system 10 is provided as described previously, wherein the phase grating 14 is configured to provide an interference pattern having an intensity peak with a full-width at half-maximum distance smaller than any value selected from the list of 0.7, 0.65, 0.60, 0.55, 0.50, 0.45, 0.4, 0.35, 0.3, 0.25, 0.2, 0.15, 0.1, 0.05, 0.04, 0.03, 0.02, or 0.01 of the period of the interference pattern.

According to an embodiment of the invention, the X-ray imaging system 10 is provided as described previously, wherein the phase grating 14 is configured to provide an interference pattern having an intensity peak with a full-width at half-maximum distance smaller than any value selected from the list of 0.7, 0.65, 0.60, 0.55, 0.50, 0.45, 0.4, 0.35, 0.3, 0.25, 0.2, 0.15, 0.1, 0.05, 0.04, 0.03, 0.02, or 0.01 of the width of the analyzer pitch W_{g}.

It is to be noted that the term “period of the interference pattern” means the distance from one point on the interference pattern, over which one full oscillation of the interference pattern's intensity has occurred.

According to an embodiment of the invention, the X-ray imaging system 10 is provided as described previously, wherein the phase grating 14 is configured to provide the interference pattern with a full-width at half-maximum distance smaller than any value selected from the list of 0.7, 0.65, 0.60, 0.55, 0.50, 0.45, 0.4, 0.35, 0.3, 0.25, 0.2, 0.15, 0.1, 0.05, 0.04, 0.03, 0.02, or 0.01 of the period of the analyzer grating.

It is to be noted that the term “period of the analyzer grating” means the distance from one point on the analyzer grating, over which one full oscillation of the analyzer grating's profile has occurred.

Therefore, an intensity peak which is narrow in comparison to a width of a transparent section of the analyzer grating may be one with dimensions selected according at least to the above definitions.

The duty cycle of the analyzer grating 16 is considered to be the represented by the ratio: width of transparent portion of analyzer grating/grating pitch.

According to an embodiment of the invention, the analyzer grating and/or phase grating have a pitch which is equal to or less than a length selected from one of the list of lengths: 0.95 μm, 1.0 μm, 1.05 μm, 1.10 μm, 1.15 μm, 1.20 μm, 1.25 μm, 1.30 μm, 1.35 μm, 1.40 μm, 1.45 μm, 1.50 μm, 1.55 μm, 1.60 μm, 1.65 μm, 1.70 μm, 1.75 μm, 1.80 μm, 1.85 μm, 1.90 μm, 1.95 μm, 2.0 μm, 2.05 μm, 2.10 μm, 2.15 μm, 2.20 μm, 2.25 μm, 2.30 μm, 2.35 μm, 2.40 μm, 2.45 μm 2.50 μm, 2.55 μm, 2.60 μm, 2.65 μm, 2.70 μm, 2.75 μm, 2.80 μm, 2.85 μm, 2.90 μm, 2.95 μm, 3.0 μm, 3.05 μm, 3.10 μm, 3.15 μm, 3.20 μm, 3.25 μm, 3.30 μm, 3.35 μm, 3.40 μm, 3.45 μm, 3.50 μm, 3.55 μm, 3.60 μm, 3.65 μm, 3.70 μm, 3.75 μm, 3.80 μm, 3.85 μm, 3.90 μm, 3.95 μm, 4.00 μm, 4.05 μm, 4.10 μm, 4.15 μm, 4.20 μm, 4.25 μm, 4.30 μm, 4.35 μm, 4.40 μm, 4.45 μm, 4.50 μm, 4.55 μm, 4.60 μm, 4.65 μm, 4.70 μm, 4.75 μm, 4.80 μm, 4.85 μm, 4.90 μm, 4.95 μm, 5.00 μm, 5.05 μm, 5.10 μm, 5.15 μm, 5.20 μm, 5.25 μm, 5.30 μm, 5.35 μm, 5.40 μm, 5.45 μm, 5.50 μm, 6.00 μm, 6.50 μm, 7.00 μm, 7.50 μm, 8.00 μm, 8.50 μm, 9.00 μm, 9.50 μm, 10.00 μm, 10.50 μm, 11.00 μm, 11.50 μm, 12.00 μm, 12.50 μm, 13.00 μm, 13.50 μm, 14.00 μm, 14.50 μm, 15.00 μm, 15.50 μm, 16.00 μm, 16.50 μm, 17.00 μm, 18.00 μm 18.50 μm, 19.00 μm, 19.50 μm, 20.00 μm.

According to an embodiment of the invention, the duty cycle of the analyzer grating and/or the phase grating is more than a value selected from the list: 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.90, 0.91, 0.92, 0.93, 0.94, 0.95.

According to an embodiment of the invention, the duty cycle of the analyzer grating and/or the phase grating lies in a range selected from the list: 0.5 to 0.9, 0.51 to 0.9, 0.52 to 0.9, 0.53 to 0.9, 0.54 to 0.9, 0.55 to 0.9, 0.56 to 0.9, 0.57 to 0.9, 0.58 to 0.9, 0.59 to 0.9, 0.6 to 0.9, 0.61 to 0.9, 0.62 to 0.9, 0.63 to 0.9, 0.64 to 0.9, 0.65 to 0.9, 0.66 to 0.9, 0.67 to 0.9, 0.68 to 0.9, 0.69 to 0.9, 0.70 to 0.9, 0.71 to 0.9, 0.72 to 0.9, 0.73 to 0.9, 0.74 to 0.9, 0.75 to 0.9, 0.76 to 0.9, 0.77 to 0.9, 0.78 to 0.9, 0.79 to 0.9, 0.8 to 0.9, 0.81 to 0.9, 0.82 to 0.9, 0.83 to 0.9, 0.84 to 0.9, 0.85 to 0.9, 0.86 to 0.9, 0.87 to 0.9, 0.88 to 0.9, 0.89 to 0.9, 0.90 to 0.96, 0.91 to 0.96, 0.92 to 0.96, 0.93 to 0.96, 0.94 to 0.96, 0.95 to 0.96.

According to an embodiment of the invention, any one of the analyzer grating/phase grating pitch lengths, and duty cycles defined above may be combined to define the width of the transparent section of the analyzer grating, and a width of the grating line 54.

As discussed above, the X-ray detector 18 may be an energy-resolving detector, such as a photon counter employing multiple energy bins. The X-ray source emits polychromatic radiation. The energy resolving detector is used to detect the attenuation or small angle scatter of incident X-rays for different energy ranges. Therefore, two independent intensity profiles may be detected by using an energy resolving detector.

According to an embodiment of the invention, the X-ray imaging system 10 as discussed above is provided, wherein the X-ray detector 18 is an energy sensitive detector configured to generate the first X-ray signal by detecting a first detected photon energy, and to generate the second X-ray signal by detecting a second detected photon energy, wherein the first and second detected photon energies are mutually different.

The following first and second photon energy ranges are applicable, for example, to a CT or X-ray system:

According to an embodiment of the invention, the first detected photon energy is in the range 25-50 keV and the second detected photon energy is in the range 50-140 keV.

According to an embodiment of the invention, the first detected photon energy is in the range 25-80 keV and the second detected photon energy is in the range 80-140 keV.

According to an embodiment of the invention, the first detected photon energy is in the range 25-100 keV and the second detected photon energy is in the range 100-140 keV.

The following first and second photon energy ranges are applicable, for example, to a mammography system:

According to an embodiment of the invention, the first detected photon energy is in the range 5-15 keV and the second detected photon energy is in the range 15-40 keV.

According to an embodiment of the invention, the first detected photon energy is in the range 5-25 keV and the second detected photon energy is in the range 25-40 keV.

According to an embodiment of the invention, the first detected photon energy is in the range 5-30 keV and the second detected photon energy is in the range 30-40 keV.

According to the above-described embodiments, two independent intensity profiles which are not affected by phase differences introduced by material inhomogeneity in the object of interest 28 can be deduced. Thus, imager reconfiguration steps, or removal/reinsertion of the object into the imager, are avoided.

Such an approach is implemented by providing a model for the energy dependent attenuation, and visibility, and spectral response of the detector. The model will depend on the specific form of the intensity profile, for example. Alternatively, or in addition, a lookup table derived by measurement of a phantom comprising different materials. For example, a phantom made of Delrin™ (being a material having a water-equivalent spectral attenuation) and a strong scattering material with negligible attenuation could be used to generate the lookup table values. The photon-counting results are mapped to the effective Delrin™ length, and scatter material length, which are then translated into the attenuation and dark-field signal.

According to an embodiment of the invention, an X-ray imaging system is provided, wherein the X-ray imaging system is configured to generate each of the first X-ray signal and the second X-ray signal as composite signals, wherein the first X-ray signal is based on a first measurement made with coherent X-rays, and a second measurement made with incoherent X-rays, and wherein the second X-ray signal is based on a third measurement made with coherent X-rays, and a fourth measurement made with incoherent X-rays, and the attenuation and dark-field components are calculated using the first measurement, second measurement, third measurement, and fourth measurement.

To generate two independent sources of information about the object of interest 28, another option is to illuminate the interferometer with coherent X-rays, to take a first set of intensity profile measurements, and then subsequently with incoherent X-rays.

According to one embodiment, the X-ray source 12 may comprise the X-ray tube 24 which emits incoherent X-ray light. The source-grating 26 makes the X-ray beam coherent. A selectable X-ray scatterer (not shown in FIG. 1) may be switched into the optical path 22 to again decohere the X-ray beam after passing through the source-grating 26.

Alternatively, an equivalent approach would be to remove the source-grating 26 from an output port of the X-ray source 12 to enable the incoherent light from the X-ray tube 24 to be applied directly to the object of interest 28.

Therefore, according to the above-described embodiments, an intensity measurement is made using the X-ray detector 18 using coherent X-rays, and then incoherent X-rays being applied.

According to an embodiment of the invention, the X-ray imaging system 10 is configured to generate the first X-ray signal by measuring the first interference pattern when an object of interest is not present in the optical path 22.

According to an embodiment of the invention, the X-ray imaging system 10 is configured to generate the first X-ray signal by measuring the second interference pattern when an object of interest is present in the optical path 22.

According to an embodiment of the invention, an X-ray imaging system is provided as discussed above, further comprising a selectable X-ray scatterer positionable in the optical path and configurable into a first state in which the X-rays are coherent, and into a second state for interacting with the X-rays such that they become incoherent; wherein the first and third measurements are made with the selectable X-ray scatterer in the first state, and wherein the second and fourth measurements are made with the selectable X-ray scatterer in the second state; and wherein the attenuation and dark-field components are calculated using the first, second, third, and fourth measurements.

The term “composite signals” refers to the fact that when generating each of the first X-ray signal, and the second X-ray signal, two measurements must be taken.

In particular, the composite signals are used to generate the first X-ray signal and the second X-ray signal according to an embodiment of the invention as follows:

According to this embodiment, four individual measurements of X-ray interference patterns are made. A pair of measurements is made without the object of interest being present in the optical path 22, and a pair of measurements is made with the object of interest present in the optical path 22.

An attenuation component (for each pixel of the X-ray detector) is defined as

A=I/I₀.

A dark field component (for each pixel of the X-ray detector) is defined as

D=V/V₀.

The “0” indicated denotes a value measured without an object being present in the optical path 22. A quantity without a subscript denotes a measurement made with an object being present in the optical path.

A model for the measured signal is, in one embodiment, signal=I(1+V).

Therefore, four single measurements are performed. A first pair is performed without an object of interest in the optical path, and a second pair is performed with an object in the optical path.

Each of the pairs of measurements are split into one measurement made with coherent X-ray radiation, and one made without incoherent radiation. As stated above, this may be achieved using a incoherent X-ray source, and switching a selectable source grating into the optical path 22 to cohere the X-ray radiation, or by providing a coherent source, and switching a scattering plate into the optical path.

Thus, the four measured signals per detector pixel can be provided as: sigh, sigIV₀, sigI and sigIV.

Now the attenuation and the dark field signal can be calculated for each detector pixel:

sigI₀=I₀ (1)

sigIV₀=I₀(1+V₀) (2)

sigI=I, (3)

sigIV=I(1+V) (4)

According to an embodiment of the invention, the X-ray imaging system as described previously is provided, wherein the X-ray detector 18 comprises a first section covered by an X-ray scatterer, and a second section not covered by the X-ray scatterer. The X-ray imaging system is configured to generate the first X-ray signal using the first section of the X-ray detector, and to generate the second X-ray signal using the second section of the X-ray detector.

According to an embodiment of the invention, a portion of the CT scanner's fan X-ray source is provided with a decohering filter, and a portion is not provided with a decohering filter.

According to these embodiments, the detection principle discussed above may be applied to a CT scanner. A combination of incoherent radiation, and coherent radiation, is provided as a result of a scattering plate placed either at the CT fan beam source, or over a portion of the CT scanner's detector.

The CT scanner detector is divided into two parts (in the fan direction, or in the z-direction). One part is provided with a strong scattering plate, and the other half is not covered. Then, for every path through the object, a projection for determining the dark-field information and another projection for determining the attenuation is provided as the CT scanner's source and detector head rotates around the patient.

As stated above, a narrow interference maximum of the intensity profile emitted from a phase grating allows most of the intensity of an incident X-ray beam to fall in the transparent section of the analyzer grating 16 having a high duty cycle (having relatively wide X-ray transparent areas, and relatively narrow blocking areas). Because the full-width at half maximum distance of the interference pattern is narrow in comparison to transparent sections of the analyzer grating 16, a phase-shift which alters the transverse position of portions of the interference pattern means that the interference maxima do not collide with the opaque gratings of the analyzer grating 16, enabling phase invariant detection.

The two independent measurements in this embodiment arise from the interference pattern gathered from the portion of the CT scanner's detector covered in the strong scattering plate, which will receive incoherent X-ray radiation, and the portion of the CT scanner's detector which is not covered in a strong scattering plate, which will receive coherent X-ray radiation.

According to an embodiment of the invention, a first set of coherent and incoherent measurements are taken by the CT scanner's detector when no object of interest is present in the optical path, and a second set of coherent and incoherent measurements are taken by the CT scanner's detector when the object of interest is positioned in the optical path.

It will be appreciated that the technique described above has a wide applicability in X-ray scanning.

According to an embodiment of the invention, the X-ray imaging system 10 is provided as previously described, wherein the X-ray imaging system is selected from the group of a CT scanner, a C-arm scanner, a mammography scanner, a tomosynthesis scanner, a diagnostic X-ray scanner, a pre-clinical imaging scanner, a non-destructive testing scanner, or a baggage security scanner.

According to an embodiment of the invention, the analyzer grating 16 is a phase-stepped analyzer grating held in a fixed position, and the phase grating 14 is configured to generate an interference pattern in the X-ray radiation comprising an intensity profile having an intensity peak with a full-width half-maximum distance which is narrow in comparison to a width of a transparent section of the analyzer grating, as described above.

It will be appreciated that the technique described above may still be applied in a conventional differential phase contrast machine with a stepped analyzer grating. The analyzer grating 16 would be held in the same position for the duration of the independent attenuation and dark-field measurements, and a special type of the phase grating 14 giving fine interference fringes would be switched into the optical path, and the conventional phase grating would be switched out of the optical path. Thus, a dual-function X-ray machine could be provided.

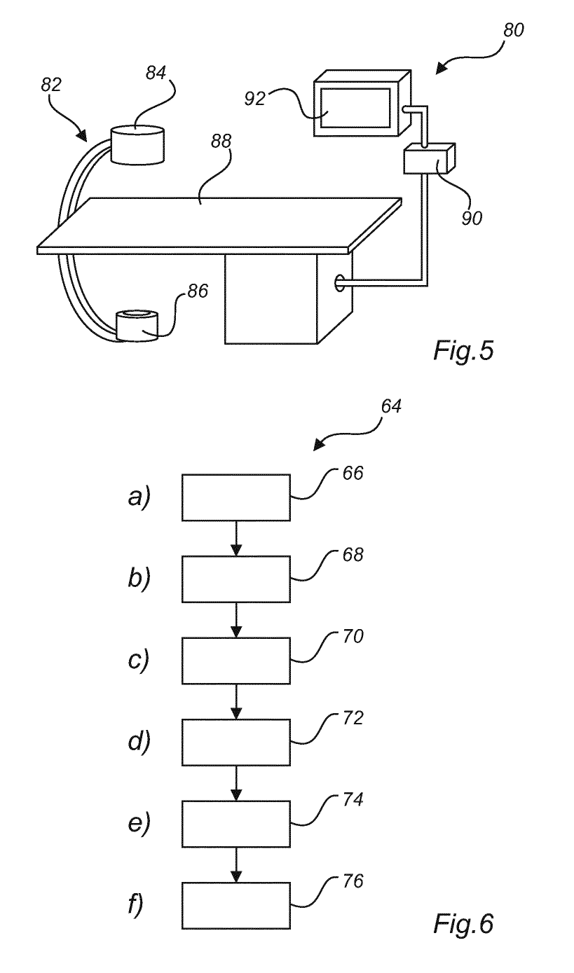

FIG. 5 illustrates a system 80 as a typical clinical application of the X-ray imaging system. The system 80 has a C-arm X-ray imager 82 comprising an X-ray source 84 and an X-ray detector 86. The X-ray source 84 may be a source as described previously in FIG. 1 comprising an X-ray tube and a source grating. The X-ray detector 86 may be a detector comprising the phase grating 14, the X-ray detector 18, and the analyzer grating 16 as described in FIG. 1. An object of interest may be placed on a table 88 in between the X-ray source 84 and in the X-ray detector 86. A processing unit 90 processes signals received from the X-ray detector 86 and an X-ray examination may be displaced on a screen 92.

According to a second aspect of the invention, a method 64 for X-ray imaging is provided, as shown in FIG. 6, the method comprises the following steps:

a) applying 66 X-ray radiation to an object of interest using an X-ray source;
b) applying 68 the X-ray radiation to a phase grating;
wherein the phase grating is configured to generate an interference pattern in the X-ray radiation comprising an intensity profile having an intensity peak with a full-width half-maximum distance which is narrow in comparison to a width of a transparent section of the analyzer grating, wherein the intensity peak of the interference pattern is incident on the X-ray detector through the transparent section of the analyzer grating;
c) applying 70 the X-ray radiation to an analyzer grating;
wherein the analyzer grating is provided in proximity to, or formed integrally with, the X-ray detector;
d) generating 72 a first X-ray signal by measuring a first interference pattern with the X-ray detector;
e) generating 74 a second X-ray signal by measuring a second interference pattern indicative of an interaction of the X-ray radiation with an object of interest in the optical path;
f) calculating 76 an attenuation component, and a dark-field component, of the first and second interference patterns using the first and second X-ray signals.

According to the second aspect of the invention, it is possible to separate the attenuation component, and the dark-field component of the applied X-rays, and therefore to provide an X-ray scanner which does not require a consecutive stepping over a complete cycle of fringe phase realizations using a mechanical grating arrangement. The complexity of an X-ray imaging method is therefore reduced.

According to an embodiment of the invention, a method is provided as described as previously, wherein in step d), the first X-ray signal is generated by detecting a first detected photon energy; and additionally in step d), the second X-ray signal is generated by detecting a second detected photon energy, wherein the first and second detected photon energies are mutually different.

According to an embodiment of the invention, the method as described above is provided, wherein in step d), the first X-ray signal is generated as a composite signal based on a first measurement made with coherent X-rays, and a second measurement made with incoherent X-rays; and wherein in step e), the second X-ray signal is also generated as a composite signal based on a third measurement made with coherent X-rays, and a fourth measurement made with incoherent X-rays.

According to an embodiment of the invention, the method as described above is provided, further comprising the steps of:

d1) switching a selectable X-ray scatterer positionable in the optical path into a first state such that the X-rays are coherent;
d2) performing the first measurement;
d3) positioning the selectable X-ray scatterer in a second state in the optical path to interact with the X-rays such that the X-rays are incoherent;
d4) performing the second measurement;
e1) positioning the selectable X-ray scatterer in a first state out of the optical path such that the X-rays are coherent;
e2) performing the third measurement;
e3) positioning the selectable X-ray scatterer in a second state in the optical path to interact with the X-rays such that the X-rays are incoherent; and
e4) performing the fourth measurement; and
wherein in step f), the attenuation and dark-field components are calculated using the first, second, third, and fourth measurements.

It will be understood by the skilled person that steps d1) to d4) and e1) to e4) may be performed in any order, provided a set of at least four measurements result (forming two composite measurements of the first X-ray signal and the second X-ray signal, respectively) in which the object of interest has been present or vacant from the optical path, and in which the X-ray beam has been incoherent or coherent.

According to an embodiment of the invention, a method is provided as described previously, wherein the phase grating is configured to generate an interference pattern having an intensity peak with a full-width half-maximum distance smaller than half of the period of the interference pattern.

According to a third aspect of the invention, a computer program element for controlling a system according to one of the previous descriptions of the X-ray system which, when being executed by a processing unit, is adapted to perform the method steps according to one of the previous methods.

According to a fourth aspect of the invention, a computer-readable medium having stored the program element previously described is provided.

According to a fifth aspect of the invention, a kit of parts for retrofitting a legacy X-ray scanner is provided.

The kit of parts comprises an X-ray detector having an analyzer grating in proximity to, or formed integrally with, the X-ray detector, and a phase grating configured to generate an interference pattern in the X-ray radiation. The phase grating comprising an intensity profile having intensity peaks with a full-width half-maximum distance which are narrow in comparison to a width of a transparent section of the analyzer grating, wherein the intensity peak of the interference pattern is incident on an installed X-ray detector through a transparent section of an analyzer grating. The kit also comprises a computer-readable medium as previously described. An installation of the kit of parts to the legacy X-ray scanner enables the legacy X-ray scanner to calculate an attenuation component, and a dark-field component, of the X-rays.

A computer program element might be stored on a computer unit which could also be an embodiment of the invention. The computing unit may be adapted to perform or induce performance of the steps of the method described above. Moreover, it may be adapted to operate the components of the above-described apparatus.

The computing unit can be adapted to operate automatically and/or to execute the orders of a user. A computer program may be loaded into a working memory or a data processor. The data processor may thus be equipped to carry out the method of the invention.

The computing unit can be supplemented with a high performance processing unit such as a graphics card, or an FPG extension card, to perform computationally intensive operations. This exemplary embodiment of the invention covers both the computer program that has the invention installed from the beginning, and a computer program that by means of an update turns an existing program into a program that uses the invention.

A computer program may be stored and/or distributed on a suitable medium, such as an optical storage media, or a solid state medium supplied together with, or as a part of other hardware, but may also be distributed in other forms, such as via the Internet or other wired or wireless telecommunication systems.

The computer program may also be presented over a network like the World Wide Web, and can be downloaded into the working memory of a data processor from such a network.

According to a further exemplary embodiment of the present invention, a medium for making a computer program element available for downloading is provided, which computer program element is arranged to perform a method according to one of the previously described embodiments of the invention.

It should to be noted that embodiments of the invention are described with reference to different subject-matters. In particular, some embodiments are described with reference to method-type claims, whereas other embodiments are described with reference to the device-type claims.

A person skilled in the art will gather from the above, and the following description that, unless otherwise notified, in addition to any combination of features belonging to one type of subject-matter, also any other combination between features relating to different subject-matters is considered to be disclosed with this application.

All features can be combined to provide a synergetic effect that is more than the simple summation of the features. While the invention has been illustrated and described in detail in the drawings and foregoing description, such illustrations and descriptions are to be considered illustrative, or exemplary, and not restrictive. The invention is not limited to the disclosed embodiments. Other variations to the disclosed embodiments can be understood, and effected by those skilled in the art in practicing the invention, from a study of the drawings, the disclosure, and the dependent claims.

In the claims, the word “comprising” does not exclude other elements or steps, and the indefinite article “a” or “an” does not exclude a plurality. A single processor, or other unit, may fulfil the functions of several items recited in the claims. The mere fact that certain measures are recited in mutually different dependent claims does not indicate that a combination of these measures cannot be used to advantage. Any reference signs in the claims should not be construed as limiting the scope.

1. An X-ray imaging system for imaging an object of interest, comprising:
an X-ray source;
a phase grating;
an analyzer grating;
an X-ray detector; and
a processing unit;
wherein the X-ray source, the phase grating, the analyzer grating, and the X-ray detector are arranged in an optical path;
wherein the X-ray source is configured to apply X-rays to an object of interest positionable in the optical path;
wherein the analyzer grating is provided in proximity to, or formed integrally with, the X-ray detector;
wherein the phase grating is configured to generate an interference pattern in the X-ray radiation comprising an intensity profile having an intensity peak with a full-width half-maximum distance which is narrow in comparison to a width of a transparent section of the analyzer grating, wherein the intensity peak of the interference pattern is incident on the X-ray detector through the transparent section of the analyzer grating;
wherein the X-ray detector is configured to generate a first X-ray signal by measuring a first interference pattern and to generate a second X-ray signal by independently measuring a second interference pattern, the interference patterns being indicative of an interaction of the X-ray radiation with an object of interest in the optical path; and
wherein the processing unit is configured to calculate an attenuation component, and a dark-field component, of the first and second interference patterns using the first and second X-ray signals,
wherein a physical characteristic of the X-ray radiation used in generating the first interference pattern and the second interference pattern is different.

2. The X-ray imaging system of claim 1,
wherein the X-ray detector is an energy sensitive detector configured to generate the first X-ray signal by detecting a first photon energy, and to generate the second X-ray signal by detecting a second photon energy, wherein the first and second photon energies are mutually different.

3. The X-ray imaging system of claim 1,
wherein the X-ray imaging system is configured to generate each of the first X-ray signal and the second X-ray signal as composite signals, wherein with the first X-ray signal is based on a first measurement made with coherent X-rays, and a second measurement made with incoherent X-rays, and wherein the second X-ray signal is based on a third measurement made with coherent X-rays, and a fourth measurement made with incoherent X-rays.

4. The X-ray imaging system of claim 3, further comprising:
a selectable X-ray scatterer positionable in the optical path and configurable into a first state in which the X-rays are coherent, and into a second state for interacting with the X-rays such that they become incoherent;
wherein the first and third measurements are made with the selectable X-ray scatterer in the first state, and wherein the second and fourth measurements are made with the selectable X-ray scatterer in the second state; and
wherein the attenuation and dark-field components are calculated using the first, second, third, and fourth measurements.

5. The X-ray imaging system of claim 1,
wherein the X-ray detector comprises a first section covered by an X-ray scatterer and a second section not covered by the X-ray scatterer; and
wherein the X-ray imaging system is configured to generate the first X-ray signal using the first section of the X-ray detector, and to generate the second X-ray signal using the second section of the X-ray detector.

6. The X-ray imaging system claim 5,
wherein the phase grating is configured to generate the interference pattern as having an intensity peak with a full-width half-maximum distance smaller than half of the period of the interference pattern.

7. The X-ray imaging system claim 6,
wherein the X-ray imaging system is selected from the group of: CT scanner, C-arm scanner, mammography scanner, tomosynthesis scanner, diagnostic X-ray scanner, pre-clinical imaging scanner, non-destructive testing scanner, or baggage security scanner.

8. A method for X-ray imaging, comprising the following steps:
a) applying X-ray radiation to an object of interest using an X-ray source;
b) applying the X-ray radiation to a phase grating;
wherein the phase grating is configured to generate an interference pattern in the X-ray radiation comprising an intensity profile having an intensity peak with a full-width half-maximum distance which is narrow in comparison to a width of a transparent section of the analyzer grating, wherein the intensity peak of the interference pattern is incident on the X-ray detector through the transparent section of the analyzer grating;
c) applying the X-ray radiation to an analyzer grating,
wherein the analyzer grating is provided in proximity to, or formed integrally with, the X-ray detector;
d) generating a first X-ray signal by measuring a first interference pattern with the X-ray detector;
e) generating a second X-ray signal by independently measuring a second interference pattern with the X-ray detector;
f) calculating an attenuation component, and a dark-field component, of the applied X-rays using the first and second X-ray signals,
wherein the first and second interference patterns are indicative of an interaction of the X-ray radiation with an object of interest in the optical path,
wherein a physical characteristic of the X-ray radiation used in generating the first interference pattern and the second interference pattern is different.

9. The method of claim 8,
wherein in step d), the first X-ray signal is generated by detecting a first photon energy;
wherein in step e), the second X-ray signal is generated by detecting a second photon energy, wherein the first and second detected photon energies are mutually different.

10. The method of claim 8,
wherein in step d), the first X-ray signal is generated as a composite signal based on a first measurement made with coherent X-rays, and a second measurement made with incoherent X-rays; and
wherein in step e), the second X-ray signal is also generated as a composite signal based on a third measurement made with coherent X-rays, and a fourth measurement made with incoherent X-rays.

11. The method of claim 8, further comprising the steps of:
d1) switching a selectable X-ray scatterer positionable in the optical path into a first state such that the X-rays are coherent;
d2) performing the first measurement;
d3) positioning the selectable X-ray scatterer in a second state in the optical path to interact with the X-rays such that the X-rays are incoherent;
d4) performing the second measurement;
e1) positioning the selectable X-ray scatterer in a first state out of the optical path such that the X-rays are coherent;
e2) performing the third measurement;
e3) positioning the selectable X-ray scatterer in a second state in the optical path to interact with the X-rays such that the X-rays are incoherent; and
e4) performing the fourth measurement; and
wherein in step f), the attenuation and dark-field components are calculated using the first, second, third, and fourth measurements.

12. The method of claim 8,
wherein in step d), the first X-ray signal is generated using a first section of the X-ray detector which is covered by an X-ray scatterer; and
wherein in step e), the second X-ray signal is generated using a second section of the X-ray detector not covered by the X-ray scatterer.

13. A computer program element for controlling a system which, when being executed by a processing unit, is adapted to perform the method steps according to claim 8.

14. A computer readable medium having stored the program element of claim 13.

15. A kit of parts for retrofitting a legacy X-ray scanner, comprising:
an X-ray detector having a static analyzer grating in proximity to, or formed integrally with, the X-ray detector;
a phase grating configured to generate an interference pattern in X-ray radiation, comprising an intensity profile having an intensity peak with a full-width half-maximum distance which is narrow in comparison to a width of a transparent section of the analyzer grating, wherein the intensity peak of the interference pattern is incident on an installed X-ray detector through a transparent section of an analyzer grating; and
a computer readable medium according to claim 14;
wherein an installation of the kit of parts to the legacy X-ray scanner enables the legacy X-ray scanner to calculate an attenuation component, and a dark-field component, of the first and second interference patterns.
